(19) United States
(12) Patent Application Publication
(10) Pub. No.: US 2004/0228765 A1
(43) Pub. Date: Nov. 18, 2004

(54) Point of care diagnostic platform

(76) Inventors: Thomas R. Witty, Santa Cruz, CA (US); Robert Case, Henderson, NV (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/745,957

(22) Filed: Dec. 23, 2003

Related U.S. Application Data
(60) Provisional application No. 60/470,725, filed on May 14, 2003.

(51) Int. Cl. G01N 33/00

(52) U.S. Cl. 422/68.1; 422/58

(57) ABSTRACT

A point of care diagnostic system has a plurality of modules and a plurality of analytic cartridges. A host computer is coupled to the plurality of modules, all of which share control by the host computer. The host computer is coupled to an interface. Each module has a corresponding interface component.

### CROSS-REFERENCE TO RELATED APPLICATIONS

This application claims the benefit of U.S. Ser. No. 60/470,725, and is related to co-pending application U.S. Ser. No. ______, (Attorney Docket No.: 40422-0003) filed the same day as the present application, all of which applications are fully incorporated herewith.

### BACKGROUND

1. Field of the Invention

The present invention relates generally to a point of care diagnostic system that has a plurality of modules and associated cartridges, and more particularly, to a point of care diagnostic system that includes a plurality of modules that share common QC protocols.

2. Description of the Related Art

Blood and other body fluid tests are important diagnostic methods in patient care and treatment. The reliability and the accuracy of the tests are critical in correctly diagnosing the patient and administrating proper treatment. The Food and Drug Administration (FDA) has established numerous quality standards for the various blood or body fluid tests. Monitoring the test process is beneficial in producing reliable and accurate test results.

One way of monitoring the test process is periodically performing the monitoring test on standard test samples. The monitoring test results are compared with expected results to verify the accuracy of the test processes or correct the test instrument or process when appropriate. In this approach, the test processes are assumed to generate consistent result between the monitoring tests.

Another way of monitoring the test process is including standard test samples in the test process. This approach is suitable for a test process that performs tests on multiple samples. The test results on the standard test samples are compared with expected results to verify the accuracy of the test processes. In this approach, the test processes on real samples are assumed to generate result consistent with those on standard test samples.

These monitoring processes are time and cost inefficient. They are deficient in meeting the needs of point of care, e.g., hospital emergency room/department, test processes. In addition to being reliable and accurate, an emergency room test process should be simple to operate and generate diversity of analytical results fast.

Accordingly, there is a need for a point of care diagnostic platform that has a plurality of modules coupled to common host computer. There is another need for a point of care diagnostic platform with a plurality of modules that share common QC protocols. Yet there is another need for a point of care diagnostic platform with a plurality of modules coupled to a host computer and an external communication system. There is still another need for a point of care diagnostic platform with a plurality of modules, and a plurality of analytic cartridges, where each cartridge is associated with a module and is configured to directly accept a blood sample from a standard blood draw tube. Yet there is a further need for a point of care diagnostic platform that has a plurality of modules, a host computer coupled to the modules, a common external communication interface, with each module sharing the common external communication interface.

### SUMMARY OF THE INVENTION

Accordingly, an object of the present invention is to provide a point of care diagnostic platform that includes a plurality of modules that share common QC protocols. (is the common operator interface another patent?)

Another object of the present invention is to provide a point of care diagnostic platform with a plurality of module coupled to a common host computer.

Yet another object of the present invention is to provide a point of care diagnostic platform with a plurality of modules, a host computer coupled to the plurality of modules and an external communication system.

Still another object of the present invention is to provide a point of care diagnostic platform with a plurality of modules, and a plurality of analytic cartridges, where each cartridge is associated with a module of the plurality of modules and is configured to directly accept a blood sample from a standard blood draw tube.

Another object of the present invention is to provide a point of care diagnostic platform with a plurality of modules; a host computer coupled to the plurality of modules and a common external communication interface, with each module sharing the common external communication interface.

A further object of the present invention is to provide a point of care diagnostic platform with a plurality of modules coupled to a common external communication interface such as a least one of WAN or a LAN.

Another object of the present invention is to provide a point of care diagnostic platform with a plurality of modules coupled to a common external communication interface that is coupled to a wireless network.

A further object of the present invention is to provide a point of care diagnostic platform with a plurality of modules coupled to a hospital information network or a laboratory information network.

Yet another object of the present invention is to provide a point of care diagnostic platform with a plurality of modules and a plurality of analytic cartridges that are each bar-coded with information for test protocols, and lot expiration dates.

Still a further object of the present invention is to provide a point of care diagnostic platform with a plurality of modules and a plurality of analytic cartridges that retain and seal fluids.

Yet another object of the present invention is to provide a point of care diagnostic platform that has a plurality of modules and a plurality of analytic cartridges, where all fluids in a cartridge, including a patient sample, remain within the cartridge.

These and other objects of the present invention are achieved in a point of care diagnostic platform. A plurality of modules are provided that share common QC protocols. A plurality of analytic cartridges are included and associated with the modules. A host computer is coupled to the plurality of modules. The host computer is coupled to an interface. Each module has a corresponding interface component.

In another embodiment of the present invention, a point of care diagnostic platform includes a plurality of modules. A host computer is coupled to the plurality of modules and a shared/common user interface. Each module is coupled to the common user interface.

In another embodiment of the present invention, a point of care diagnostic platform includes a plurality of modules. A host computer is coupled to the plurality of modules and an external communication system.

### BRIEF DESCRIPTION OF THE DRAWINGS

FIG. 1(a) is a block diagram illustrating one embodiment of a point of care diagnostic platform of the present invention, with a user interface, host computer, multiple single-cartridge test processing modules and an external communication system.

FIG. 1(b) is a block diagram illustrating another embodiment of a point of care diagnostic platform of the present invention, with multiple multi-cartridge test processing modules.

FIG. 1(c) is a block diagram illustrating another embodiment of a point of care diagnostic platform of the present invention, with the host computer being integrated with multiple, multi-cartridge modules.

FIG. 1(d) is a block diagram illustrating another embodiment of a point of care diagnostic platform of the present invention, with the host computer and user interface both integrated with multiple, multi-cartridge modules.

FIG. 1(e) is a block diagram illustrating another embodiment of a point of care diagnostic platform of the present invention, with the host computer and user interface integrated with multiple, single-cartridge modules.

FIG. 2 is a cross-sectional view of one embodiment of a cartridge that can be utilized with the point of care diagnostic platform of the present invention.

FIG. 3 is a cross-sectional view of a sample tube that can be utilized with cartridges of the present invention.

FIG. 4 is a schematic diagram illustrating one embodiment of the docking, and the relationship between a cartridge and a module of the present invention.

FIG. 5 is a schematic diagram illustrating another embodiment of the docking, and the relationship between a cartridge and a module of the present invention.

FIG. 6 is a schematic diagram illustrating another embodiment of the docking, and the relationship between a cartridge and a module of the present invention.

FIG. 7 is a cross-sectional view of one embodiment of a cartridge utilized with the present invention, illustrating air, sample and reagent flow channels.

FIG. 8 is a flow chart illustrating an overall methodology of the point of care diagnostic platform of the present invention.

FIG. 9 is a flow chart illustrating one embodiment of a cartridge processing procedure implemented with the point of care diagnostic platform of the present invention.

FIG. 10 is a flow chart illustrating one embodiment of an immunoassay operating procedure implemented with the point of care diagnostic platform of the present invention.

FIG. 11 is a flow chart illustrating one embodiment of a hematology operating procedure implemented with the point of care diagnostic platform of the present invention.

### DETAILED DESCRIPTION OF THE PREFERRED EMBODIMENT

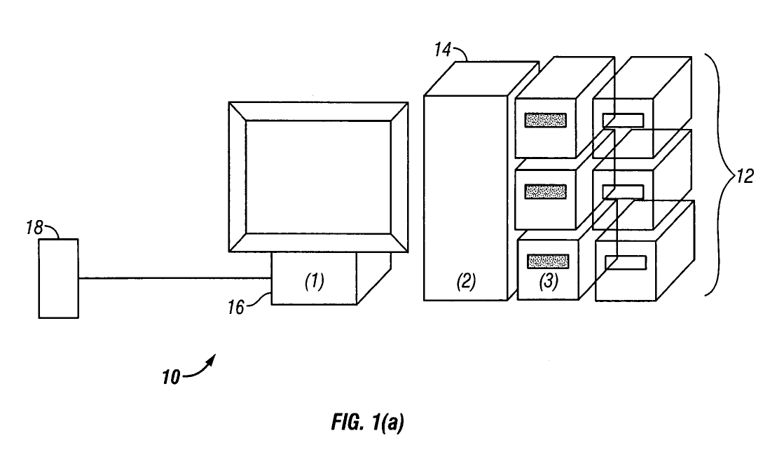

As illustrated in FIG. 1(a), one embodiment of the present invention is a point of care diagnostic platform, denoted generally as 10, and its method of use. Point of care diagnostic platform 10 includes a plurality of modules 12. A variety of different modules can be included but not limited to, immunoassay, hematology, electrolyte, molecular diagnostic, coagulation, blood gas, chemistry and the like. The modules 12 can share at least a portion of a common functionality of operation such as fluid movement, sample introduction, and the like. In one embodiment, each module 12 contains common functionalities, and unique technologies that correspond to one or more selected chemistries. In the FIG. 1(a) embodiment, modules 12 are multiple single-cartridge test processing modules.

Platform 10 can deliver a multitude of discreet testing capabilities in a standardized manner. Modules 12 can have common operation platforms. Examples of common operation systems are user interface, quality control, calibration, training, connection to various laboratory information systems, hospital information systems, emergency room information systems, wireless communication and the like.

A host computer 14 is coupled to the plurality of modules 12 and also to a user interface 16. Each module 12 is coupled to the user interface 16. Host computer 14 has a variety of different capabilities, including but not limited to user interface, quality control, calibration, training, connection to various laboratory information systems, hospital information systems, emergency room information systems, wireless communication and the like. User interface 16 is coupled to each module 12 User interface 16 provides uniform (automated and standardized) connectivity to the plurality of modules 12 as well as communication to other hospital and laboratory information systems. It will be appreciated that standardized includes industry standards as documented by the Connectivity Industry Consortium. User interface 16 establishes a database of analyzed samples and provides the operator with quality control options for the plurality of modules 12. This is achieved by centralizing and tracking the collective output of the plurality of modules 12. In one embodiment, user interface 16 includes capability for at least one of a cardiac, fertility, kidney, coagulation, electrolyte and hematology panel, molecular diagnostics and chemistry panels, and the like.

Each module 12 has a corresponding interface component for module control and sample results acquisition. In one embodiment, host computer 14 is also coupled to an external communication system 18. A variety of different external communication systems are suitable including but not limited to a, WAN, LAN, wireless network, hospital information network, laboratory information network, and the like. Platform 10 can be connected directly or in-directly to a emergency room/department patient management network

In one embodiment, each module 12 shares common QC protocols. The QC protocols include but are not limited to the following, module electronic verification, real-time process monitoring, patient record-keeping, periodic liquid control results monitoring, and the like. The QC protocols are initiated in the same manner regardless of the module 12 that is tested. Electronic monitoring of the process at each module 12 is continuous and transparent to the operator and do not require operator attention. Results are stored in module specific databases. Each module can utilize specific electronic and/or optical parameter monitoring. Changes in the electronic and optical parameters are tracked during the operation of the module 12 involved, and the outputs compared to expected thresholds/changes. These changes are indicative of correct internal operation during sample processing.

In another embodiment, illustrated in FIG. 1(b), multiple, multi-modules are provided, where a module 12 can be utilized with more than one cartridge. In FIG. 1(c) host computer 14 is integrated with multiple, multi-cartridge modules 12. In the FIG. 1(d) embodiment, host computer 14 and user interface 16 are both integrated with multiple, multi-cartridge modules 12. In the FIG. 1(e) embodiment, host computer and user interface 16 are integrated with multiple, single-cartridge test processing modules 12.

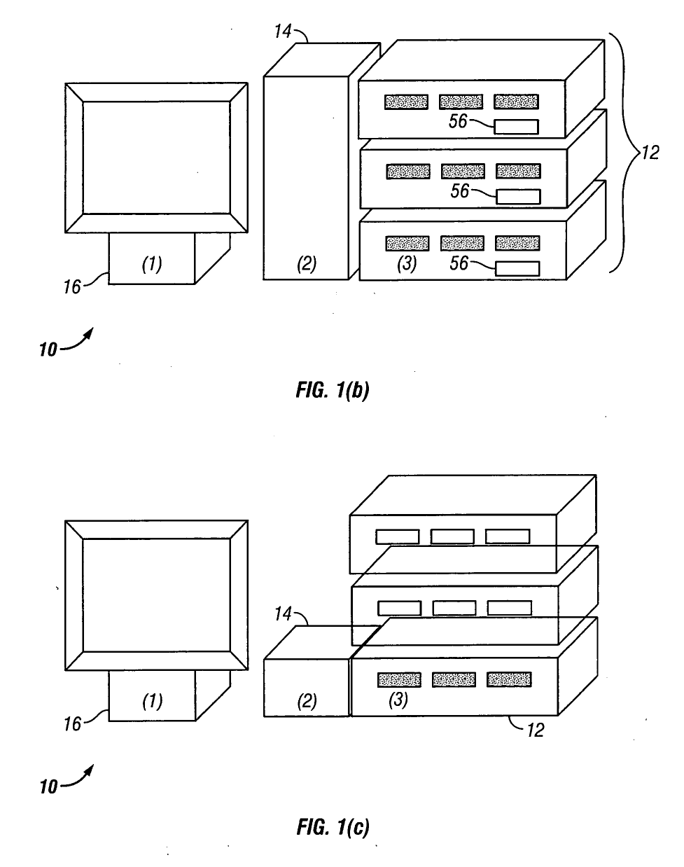

Point of care diagnostic platform 10 includes a plurality of cartridges 20, illustrated in FIG. 2. Cartridges 20 include but are not limited to cardiac, fertility, kidney, coagulation, electrolyte and hematology panel, molecular diagnostics and chemistry panels, and the like.

Each cartridge 20 can include a dock 22 for receiving a sample tube, an air dock 24 that can be engaged by a module 12, a rotary valve 26, which can also be engaged by a module 12, a calibration chamber 28, waste chamber 30, sample/calibration flow path 32 which is coupled to a detector, sample out flow 34, sample pressure channel 36 and a flow cell 38 which is a detection chamber.

Cartridges 20 can have wet and dry chemistries and at least one substrate that carriers a chemistry. Examples of various wet and dry chemistries are listed in table 1.

TABLE 1

| cartridge | Wet Reagents | Dry Reagents |
|---|---|---|
| electrolytes | calibration fluid | ion specific electrode |
| immunology | — | Capture antibody |
| | | Conjugate antibody |
| hemolotogy | Lysing solution/white blood cell | — |
| | nuclear label | |
| | Hemoglobin dye | |
| Chemistry | Various | Various |
| Coagulation | — | Initiator |
| Blood gas | — | Electrode |
| Molecular | Nucleic acid label | Nucleic acid capture |
| | | Amplification reagents |

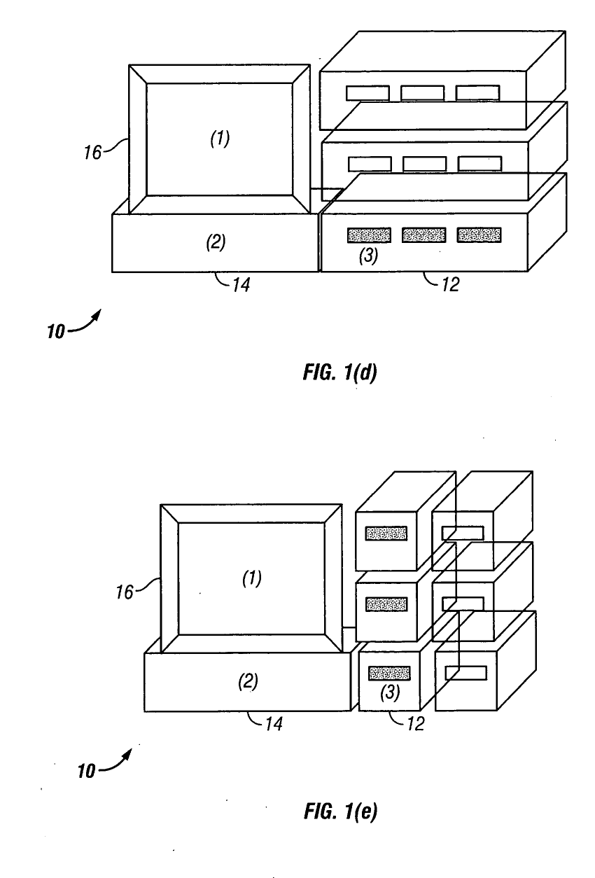

Cartridges 20 are associated with a corresponding module 12. In one embodiment, cartridges 20 can directly accept a blood sample from a standard blood draw, sample tube 40 which can include a pressure needle 42 and a sampling needle 44, as shown in FIG. 3. This can be achieved by, (i) piercing the cap of the standard blood draw tube 40 needles 42 and 44, which deliver low pressure air to force the sample through the other needle into the cartridge 20, penetrating the cap with a single needle and withdrawing fluid directly using a vacuum, and the like. Cartridges 20 can be configured to retain and seal fluids. This can be achieved by using selective pressurization of reagent and sample reservoirs, which forces the fluids into cartridges 20 and through flow cell 38 into waste chamber 30, that can be an integral part of cartridges 20. All fluids in cartridges 20, including patient samples, can remain within the cartridge 20.

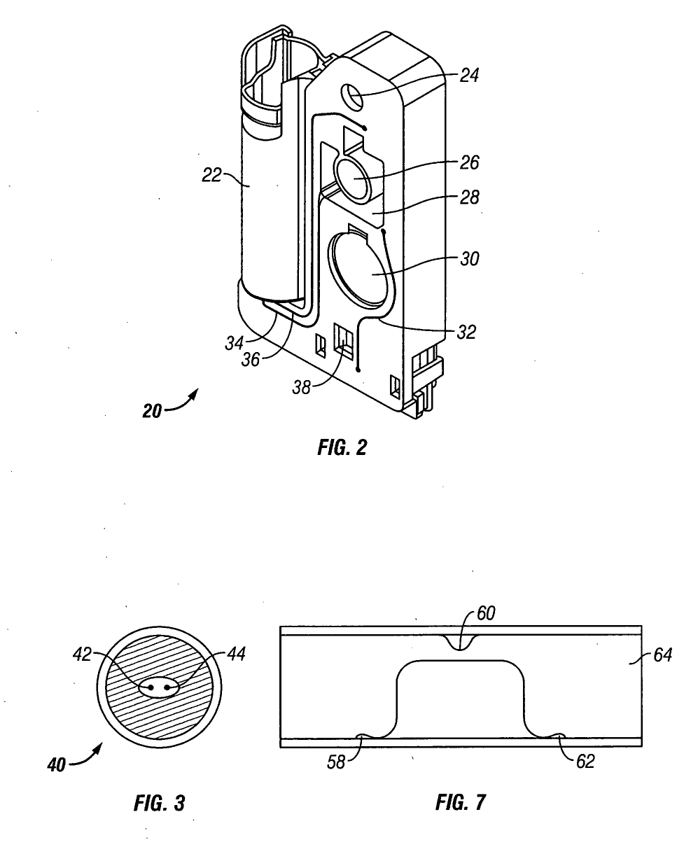

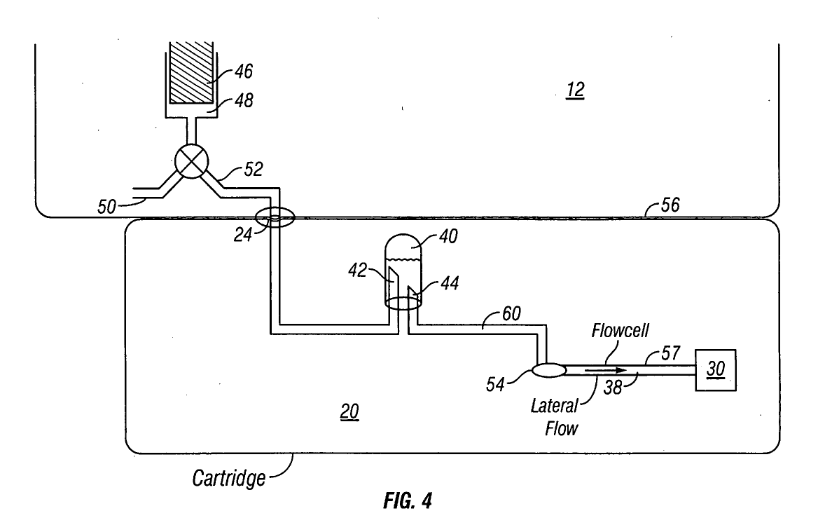

As illustrated in FIG. 4, modules 12 can be configured to be engaged with the cartridges 20 to produce pneumatic movement of fluids in the cartridges 20. The pneumatic pressure is applied by an external pump 46 through the dock 22 on cartridge 20, FIG. 2, which is engaged by module 12. Module 12 can include a valve, 48, a vent 50 to atmosphere and a channel 52 that is coupled to cartridge 20. The pneumatic pressure is directed to specific reservoirs and samples in cartridge 20 using valve 48 mechanism to cause selective reagent flow. Cartridge 20 includes a sample application area 54. Optics 56 are included in module 12 and an optical window 57 is included in cartridge 20. At the cessation of reagent flow, excess pressure is vented through vent 50 to atmosphere to stop the flow. Platform 10 can provide self-testing of modules 12, to provide for monitoring and detection of fluid flow. Various electrical and optical properties of the samples and reagents allow continuous monitoring of flow cell contents and are compared to expected transition values, as illustrated in FIG. 5.

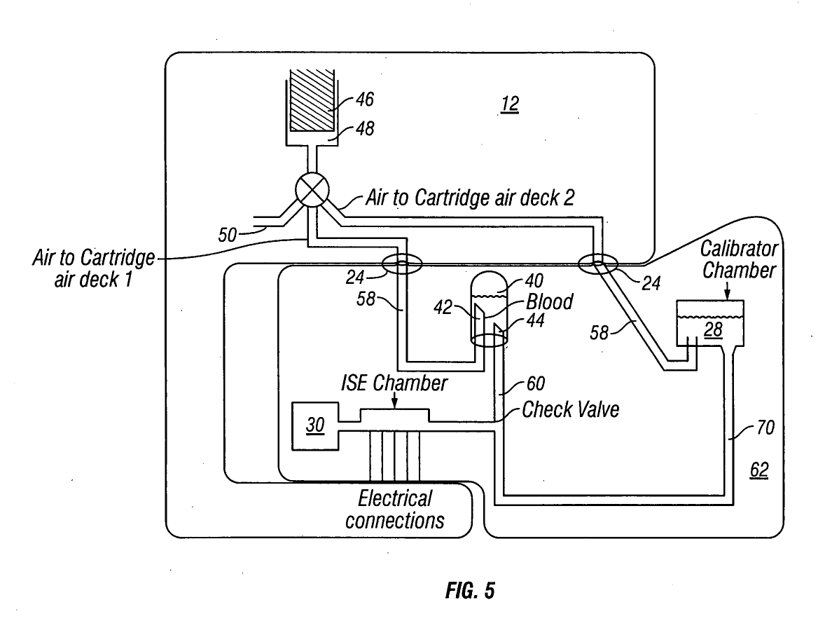

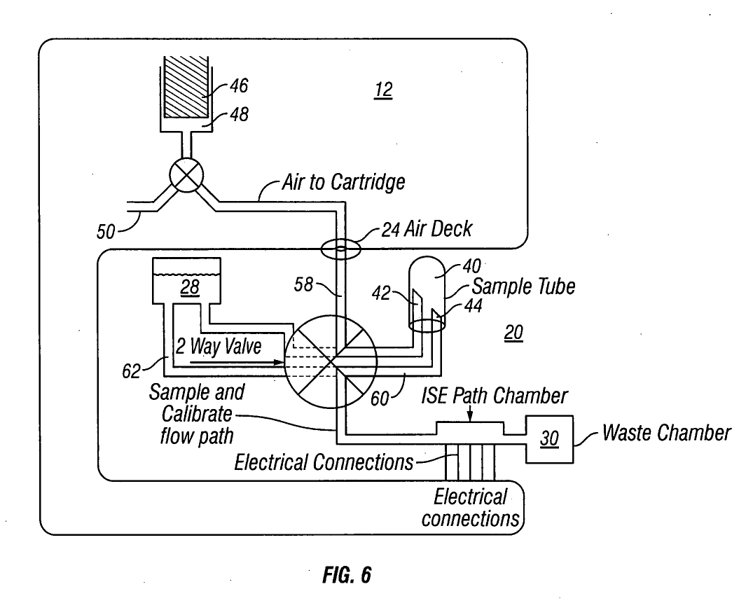

FIG. 6 illustrates a cross-sectional view of one embodiment of a cartridge 20. Cartridge 20 can have a number of different flow channels, including but not limited to air, sample and reagent flow channels 58, 60 and 62. Flow channels 58-62 can be created by depressions in both the top and bottom surfaces of the cartridge 20. Flow paths 58-62 can then be sealed with a vapor barrier 64.

Referring again to FIG. 4, pressurization of specific sample or reagent containers provided by pump 46 are selectively directed to sample and reagents containers in sequence, providing an outflow directed by a valve to detection chamber 38 or other location, as needed, in sequence and with precise timing. The sample and reagents can flow through an area of controlled temperature to prepare them for precise analysis prior to or during introduction to detection chamber 38. After analysis the reagents and sample remain in the cartridge 20 in waste region 30, although the sample tube 40 can be removed by the operator for subsequent use if desired.

Each module 12 can include a processor 56 (FIG. 1(b). Host computer 16, in combination with a processor 56, determines a test protocol for a cartridge 20. A fluid control mechanism in the cartridge 20 is then actuated that permits a flow of a patient sample with liquid chemistries and waste materials. This can occur without exposing an operator of platform 10 and the patient, to a transfer of a patient sample into the cartridge 20 without exposure to the chemistries. Cartridges 20 are designed to isolate biohazards in a cartridge 20 from an operator of the cartridge 20 or the patient. Blood samples from patients are introduced to the cartridges 20 while isolating biohazards in the cartridge from an operator.

In one embodiment, cartridges 20 are designed to work with whole blood. This eliminates the requirement of a secondary process to remove the cellular components which may interfere with the testing. This additional separation is both time consuming and error prone. In the cartridge, the separation of cells is done automatically by providing a barrier which is penetrated by the analyte to be measured by excludes the cells from analytical contact, except in the case of hemotalogy, where the cells themselves are the subject of measurement.

Cartridges 20 can include electronic identifiers, including but not limited to bar-coded identifiers, with information for test protocols, and lot expiration dates. Cartridges 20 can also include serialized identification.

In one embodiment, placement of a cartridge 20 in a module 12 begins an initiation of the module 12. When a cartridge 20 is inserted into a module 12 it can be sensed automatically. The bar code of cartridge 20, with its unique sample, are read. This initiates the sequential operation of the fluid movement and detection.

In another embodiment of the present invention, platform 10 includes a plurality of modules 12 each sharing common QC protocols. A list of possible QC protocols is found in table 2.

TABLE 2

| | |
|---|---|
| Responsibility | Comments |

Model POCT Platform Operating Procedure

| | | | |
|---|---|---|---|
| | Action (per cartridge) | | |
| 1. | Draw minimum of 1.5 ml whole blood | Operator | Exact volume |
| | sample in appropriate 5 ml vacutainer-type | | above minimum not |
| | draw tube, using standard draw procedure | | critical |
| 2. | Push sample tube into cartridge tube dock | Operator | |
| | and fully seat over needles | | |
| 3. | Place patient ID bar code label in | Operator | If ED bar code |
| | designated target area on tube dock | | system used |
| 4. | Push cartridge into module port until fully | Operator* | Platform in testing |
| | seated over snap-type detents | | mode |
| 5. | LED (blue) above port flashes to indicate | Platform | No LED, push |
| | cartridge fully seated in port and cartridge | | cartridge further |
| | read in process | | into port |
| 6. | LED steady illumination after 2 seconds if | Platform | No steady LED, |
| | cartridge read OK (lot#, exp. Date, test | | replace cartridge |
| | type, patient ID) OPERATOR WALK AWAY | | and reuse sample |
| 7. | Perform designated assay protocol | Platform | 10-15 minutes |
| 8. | LED extinguishes, patient, test results, | Platform | Downloaded to LIS |
| | reference range and QC data stored in | | when connected |
| | memory, displayed on screen and printed | | |
| | on attached printer | | |
| 9. | Remove cartridge and discard in | Operator* | |
| | biohazardous solid waste (remove and sale | | |
| | sample tube if required) | | |

Immunoassay

| | | | |
|---|---|---|---|
| 1. | Action | | |
| | Draw minimum of 1.5 ml whole blood | Operator | Exact volume |
| | sample in appropriate 5 ml vacutainer-type | | above minimum not |
| | draw tube, using standard draw procedure | | critical |
| 2. | Push sample tube into cartridge tube dock | Operator | |
| | and fully seat over needles | | |
| 3. | Place patient ID bar code label in | Operator | If ED bar code |
| | designated target area on tube dock | | system used |
| 4. | Push cartridge into module port until fully | Operator | |
| | seated over detents | | |
| 5. | Pressurize sample tube and flow sample: | Plafform | 3X volumes for (3) |
| | 200 ul/test trip at 500 u./min. | | strip cartridge |
| 6. | Stop flow by: | Platform | Test strip manifold |
| | a. venting pressure to test strip manifold, or | | is a porous |
| | b. flow channel manifold if even distribution | | membrane |
| 7. | Read reflectance change on strip reaction | Platform | |
| | areas at designated intervals | | |

Hemotology

| | | | |
|---|---|---|---|
| 1. | Draw minimum of 1.5 ml whole blood | Operator | Exact volume |
| | sample in appropriate 5 ml vacutainer- | | above minimum not |
| | type draw tube, using standard draw | | critical |
| | procedure | | |
| 2. | Push sample tube into cartridge tube | Operator | |
| | dock and fully seat over needles | | |
| 3. | Place patient ID bar code label in | Operator | If ED bar code |
| | designated target area on tube dock | | system used |
| 4. | Push cartridge into module port until fully | Operator | LED illuminates |
| | seated over detents | | above port |
| 5. | Pressurize sample tube and flow sample | Platform | |
| | to segment at 200 ul sample | | |
| 6. | Stop flow by venting pressure to sample | Platform | |
| | tube | | |
| 7. | Pressurize diluent and flow to wash | Platform | |
| | sample segment into mixing chamber | | |
| 8. | Stop flow by venting pressure | Platform | |
| 9. | Mix sample and diluent | Platform | How mix? |
| 10. | Pressurize mixed sample and flow to | Platform | |
| | flowcell. | | |
| 11. | Stop flow by venting pressure | Platform | |
| 12. | Repeat steps 10 and 11 (4) times | Platform | |
| 13. | Segment 50 ul of sample | Platform | |
| 14. | Mix with 500 ul of Hb reagent | Platform | |
| 15. | Flow mixed sample into flowcell: 100 ul at | Platform | |
| | 1 ml/min | | |

Electrolytes

| | | | |
|---|---|---|---|
| 1. | Draw minimum of 1.5 ml whole blood | Operator | Exact volume |
| | sample in appropriate 5 ml vacutainer- | | above minimum not |
| | type draw tube, using standard draw | | critical |
| | procedure | | |
| 2. | Push sample tube into cartridge tube dock | Operator | |
| | and fully seat over needles | | |
| 3. | Place patient ID bar code label in | Operator | If ED bar code |
| | designated target area on tube dock | | system used |
| 4. | Push cartridge into module port until fully | Operator | |
| | seated over detents | | |
| 5. | Pressurize sample tube and flow sample | Platform | |
| | to segment: 300 ul at 2 ml/min. | | |
| 6. | Stop flow by venting pressure | Platform | |
| 7. | Pressurize sample tube and flow sample | Platform | |
| | through cartridge: 400 ul at 3 ml/min. | | |
| 8. | Stop flow by venting pressure | Platform | |

*Operation at Instrument

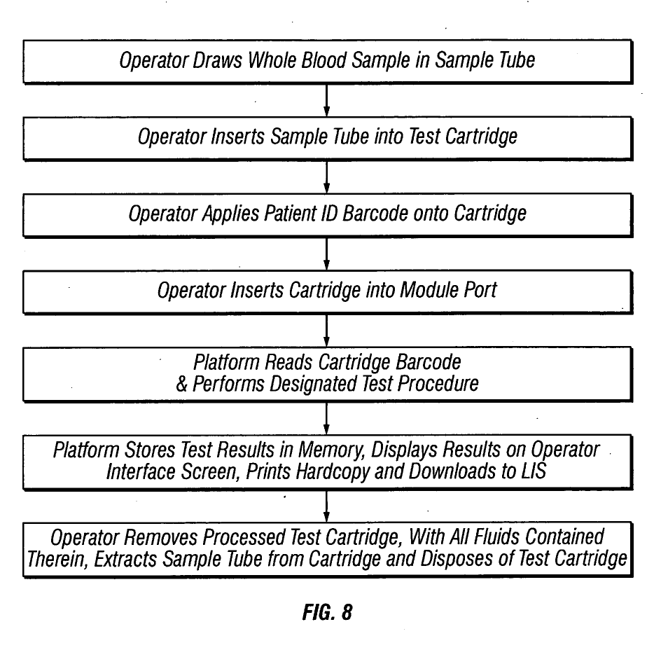

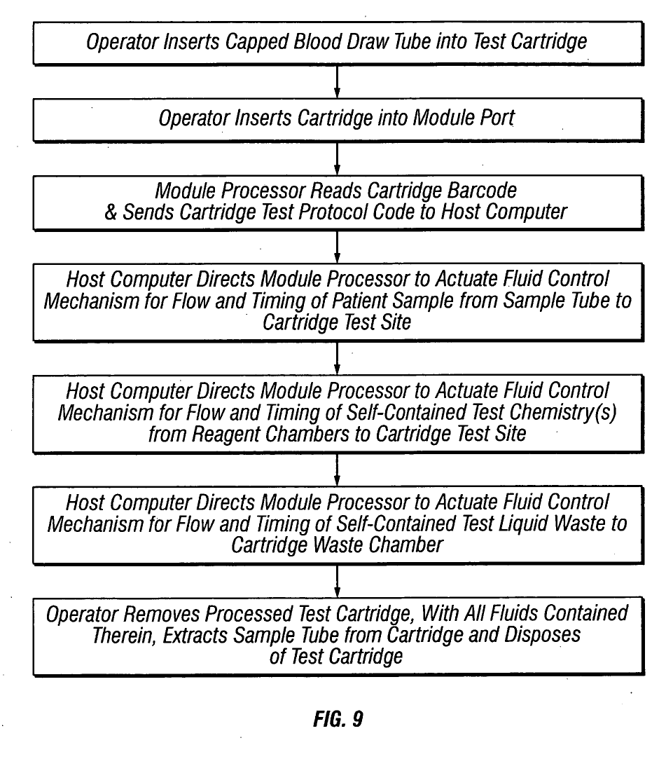

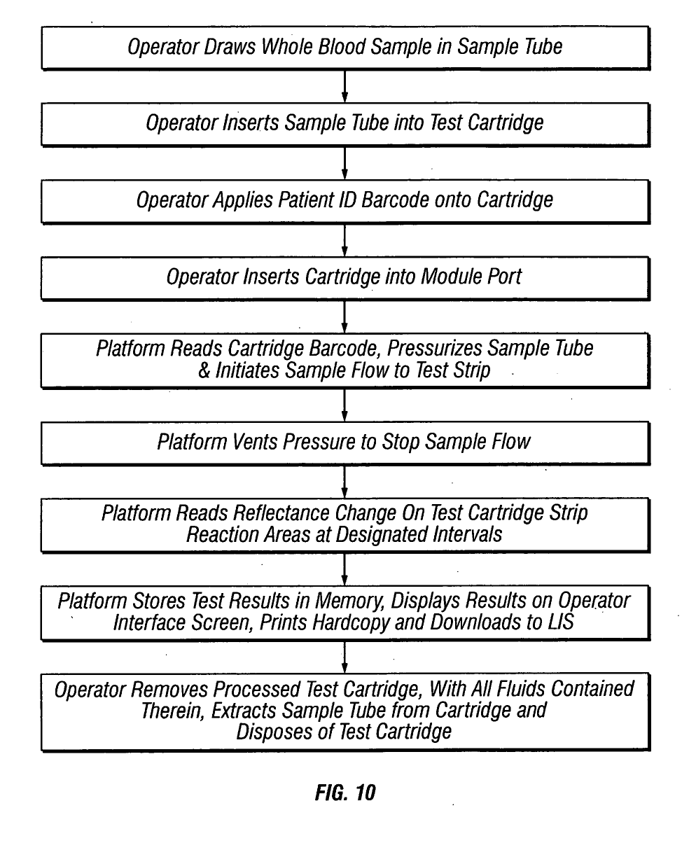

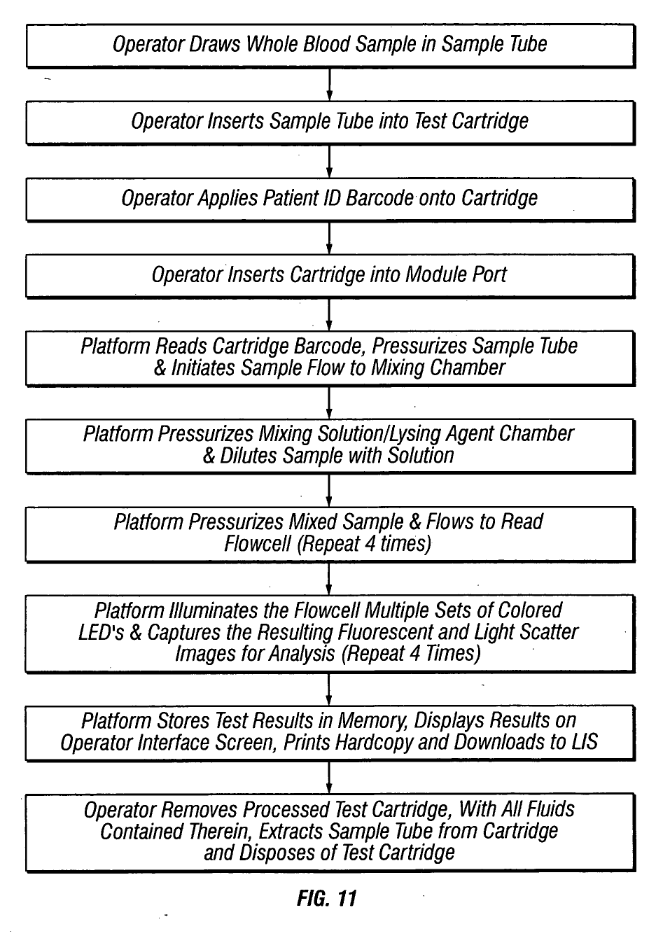

FIG. 8 through 11 are flow charts illustrating point of care diagnostic platform 10 of the present invention. FIG. 8 is a flow chart illustrating an overall methodology of the point of care diagnostic platform of the present invention. FIG. 9 is a flow chart illustrating one embodiment of a cartridge processing procedure implemented with the point of care diagnostic platform of the present invention. FIG. 10 is a flow chart illustrating one embodiment of an immunoassay operating procedure implemented with the point of care diagnostic platform of the present invention. FIG. 11 is a flow chart illustrating one embodiment of a hematology operating procedure implemented with the point of care diagnostic platform of the present invention.

In the preceding example, all reagents and waste are contained in cartridge 20. Fluids are moved in cartridge 20 via an external pump (in the module) coupled to cartridge 20 via an air dock. Likewise the reagents and sample are directed sequentially by valve(s) with-in the cartridge but activated through physical engagement to an external activator in the module. Cartridge 20 contains the fluid flow, fluid distribution fluid segmentation and sample dilution. A module 12 controls the fluid flow via a low pressure air connection and the fluid selection via one or more valve connections.

In another embodiment, platform 10 provides real time QC monitoring, and real time test result threshold detection, as disclosed in U.S. Provisional No. 60/470,725, incorporated herein by reference.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A point of care diagnostic system, comprising:
a plurality of modules, each module sharing common QC protocols;
a plurality of analytic cartridges; and
a host computer coupled to the plurality of modules, the host computer being coupled to an interface, each of a module having a corresponding interface component.

2. The system of claim 1, wherein the system includes immunoassay, hematology, electrolyte, general chemistry and molecular diagnostic modules.

3. The system of claim 1, wherein the host computer is coupled to at least one external communication link.

4. The system of claim 3, wherein the external communication link is a WAN or a LAN.

5. The system of claim 3, wherein the external communication link is a wireless network.

6. The system of claim 3, wherein the external communication link is a hospital information network or a laboratory information network.

7. The system of claim 1, wherein each of the modules share at least a portion of a common functionality of operation.

8. The system of claim 7, wherein placement of a cartridge in a module begins an initiation of the module.

9. The system of claim 1, wherein each module contains common functionalities, and unique technologies that correspond to one or more selected chemistries.

10. The system of claim 1, wherein each cartridge is bar-coded.

11. The system of claim 10, wherein each cartridge is bar-coded with information for test protocols, and lot expiration dates.

12. The system of claim 1, wherein fluids are retained and sealed in the cartridges.

13. The system of claim 1, wherein the cartridges include serialized identification.

14. The system of claim 12, wherein a sample ID barcode is attached to the cartridge for automatic patient identification.

15. The system of claim 12, wherein a sample contained in a sample tube is removable after analysis.

16. The system of claim 12, wherein all fluids in a cartridge remain within the cartridge including a patient sample.

17. The system of claim 1, wherein each cartridge includes wet and dry chemistries and at least one substrate that carriers a chemistry.

18. The system of claim 1, wherein the modules are configured to be engaged with cartridges to produce pneumatic movement of fluids in the cartridges.

19. The system of claim 1, wherein each module includes a processor.

20. The system of claim 19, wherein a processor of a module in combination with the host computer determines a test protocol for a cartridge and actuates a flow control mechanism in the cartridge that permits a flow of a patient sample with liquid chemistries and waste materials.

21. The system of claim 19, wherein a processor of a module in combination with the host computer activates a transfer of a sample into the cartridge without exposing an operator to the chemistry.

22. The system of claim 1, wherein each cartridge is configured to isolate biohazards in the cartridge from an operator of the cartridge.

23. The system of claim 1, wherein each cartridge is configured to provide introduction of a blood sample to the cartridge while isolating biohazards in the cartridge from an operator.

24. The system of claim 1, wherein each cartridge is configured to provide introduction of a blood sample to the cartridge without exposing an operator to patient material in the cartridge.

25. The system of claim 1, wherein each cartridge is configured to provide removal of a blood sample from a vial and introduction of the blood sample to the cartridge without exposing the patient or an operator to the blood sample.

26. The system of claim 1, wherein each cartridge is configured to provide that an operator is not exposed to contents in the cartridge.

27. The system of claim 1, wherein the cartridge is configured to work with whole blood.

28. The system of claim 1, wherein the system is connected to a laboratory information system.

29. The system of claim 1, wherein the system is directly connected to a laboratory information system.

30. The system of claim 1, wherein the system is connected to a hospital information system.

31. The system of claim 1, wherein the system is directly connected to a hospital information system.

32. The system of claim 1, wherein the system is connected to a emergency room/department patient management network.

33. The system of claim 1, wherein the system is directly connected to a emergency department patient management network.

34. The system of claim 1, wherein the host computer is coupled to at least one of a LAN, a WAN and a wireless network.

35. The system of claim 1, wherein the system delivers a multitude of discreet testing capabilities in a standardized manner.

36. The system of claim 1, wherein the modules have common operation systems.

37. The system of claim 1, wherein the communication interface includes at least one of a cardiac, fertility, kidney, coagulation, electrolyte and hematology panel.

38. The system of claim 1, wherein each cartridge is configured to directly accept directly a blood sample from a standard blood draw tube.

39. The system of claim 1, wherein each cartridge direct sampling, uses primary tube direct sampling which is directly introduced to the cartridge.

40. The system of claim 1, further comprising:
a coagulation module; and
a coagulation cartridge.

41. The system of claim 1, wherein the system provides real time QC monitoring, and real time test result threshold detection.

42. The system of claim 1, wherein the cartridges include electronic identifiers

43. The system of claim 1, wherein the system provides self-testing of the modules, to provide for monitoring and detection of fluid flow.

44. A point of care diagnostic system, comprising:
a plurality of modules; and
a host computer coupled to the plurality of modules and an operator interface, each of the modules coupled to the user interface.

45. A point of care diagnostic system, comprising:
a plurality of modules; and
a host computer coupled to the plurality of modules and an external communication system.

46. The system of claim 45, wherein the system includes immunoassay, hematology, electrolyte, general chemistry and molecular diagnostic modules.

47. The system of claim 46, wherein the external communication link is a wireless network.

48. The system of claim 46, wherein the external communication link is a hospital information network or a laboratory information network.

49. The system of claim 45, wherein each of the modules share at least a portion of a common functionality of operation.

50. The system of claim 50, wherein placement of a cartridge in a module begins an initiation of the module.

51. The system of claim 45, wherein each module contains common functionalities, and unique technologies that correspond to one or more selected chemistries.

52. The system of claim 51, wherein each cartridge is bar-coded.

53. The system of claim 53, wherein each cartridge is bar-coded with information for test protocols, and lot expiration dates.

54. The system of claim 51, wherein fluids are retained and sealed in the cartridges.

55. The system of claim 51, wherein the cartridges include serialized identification.

56. The system of claim 55, wherein a sample ID barcode is attached to the cartridge for automatic patient identification.

57. The system of claim 55, wherein a sample contained in a sample tube is removable after analysis.

58. The system of claim 55, wherein all fluids in a cartridge remain within the cartridge including a patient sample.

59. The system of claim 51, wherein each cartridge includes wet and dry chemistries and at least one substrate that carriers a chemistry.

60. The system of claim 51, wherein the modules are configured to be engaged with cartridges to produce pneumatic movement of fluids in the cartridges.

61. The system of claim 45, wherein each module includes a processor.

62. The system of claim 62, wherein a processor of a module in combination with the host computer determines a test protocol for a cartridge and actuates a flow control mechanism in the cartridge that permits a flow of a patient sample with liquid chemistries and waste materials.

63. The system of claim 62, wherein a processor of a module in combination with the host computer activates a transfer of a sample into the cartridge without exposing an operator to the chemistry.

64. The system of claim 51, wherein each cartridge is configured to isolate biohazards in the cartridge from an operator of the cartridge.

65. The system of claim 51, wherein each cartridge is configured to provide introduction of a blood sample to the cartridge while isolating biohazards in the cartridge from an operator.

66. The system of claim 51, wherein each cartridge is configured to provide introduction of a blood sample to the cartridge without exposing an operator to patient material in the cartridge.

67. The system of claim 51, wherein each cartridge is configured to provide removal of a blood sample from a vial and introduction of the blood sample to the cartridge without exposing the patient or an operator to the blood sample.

68. The system of claim 51, wherein each cartridge is configured to provide that an operator is not exposed to contents in the cartridge.

69. The system of claim 51, wherein the cartridge is configured to work with whole blood.

70. The system of claim 45, wherein the system is connected to a laboratory information system.

71. The system of claim 45, wherein the system is directly connected to a laboratory information system.

72. The system of claim 45, wherein the system is connected to a hospital information system.

73. The system of claim 45, wherein the system is directly connected to a hospital information system.

74. The system of claim 45, wherein the system is connected to a emergency room/department patient management network.

75. The system of claim 45, wherein the system is directly connected to a emergency department patient management network.

76. The system of claim 45, wherein the host computer is coupled to at least one of a LAN, a WAN and a wireless network.

77. The system of claim 45, wherein the system delivers a multitude of discreet testing capabilities in a standardized manner.

78. The system of claim 45, wherein the modules have common operation systems.

79. The system of claim 45, wherein the communication interface includes at least one of a cardiac, fertility, kidney, coagulation, electrolyte and hematology panel.

80. The system of claim 51, wherein each cartridge is configured to directly accept directly a blood sample from a standard blood draw tube.

81. The system of claim 51, wherein each cartridge is configured to provide direct sampling that uses a primary tube direct sampling that is directly introduced to the cartridge.

82. The system of claim 45, further comprising:
a coagulation module; and
a coagulation cartridge.

83. The system of claim 45, wherein the system provides real time QC monitoring, and real time test result threshold detection.

84. The system of claim 45, wherein the cartridges include electronic identifiers

85. The system of claim 45, wherein the system provides self-testing of the modules, to provide for monitoring and detection of fluid flow.
